(19) United States
(12) Patent Application Publication
(10) Pub. No.: US 2007/0185072 A1
(43) Pub. Date: Aug. 9, 2007

(54) Nitrogeneous polycyclic derivatives useful as chelators of metal ions and their applications

(76) Inventors: Christophe Boldron, Toulouse (FR); Marguerite Pitie, Ramonville (FR); Bernard Meunier, Castanet (FR)

Correspondence Address: NIXON & VANDERHYE, PC, 901 NORTH GLEBE ROAD, 11TH FLOOR, ARLINGTON, VA, 22203, US

(21) Appl. No.: 10/550,143

(22) PCT Filed: Mar. 22, 2004
(86) PCT No.: PCT/EP04/04016

§ 371 Date: Feb. 6, 2007

Related U.S. Application Data
(60) Provisional application No. 60/456,246, filed on Mar. 21, 2003.

(51) Int. Cl. A61K 31/555; A61K 31/4745

(52) U.S. Cl. 514/185; 514/279

(57) ABSTRACT

The invention relates to the use of nitrogeneous polycyclic derivatives for preparing drugs for treating neurodegenerative diseases,

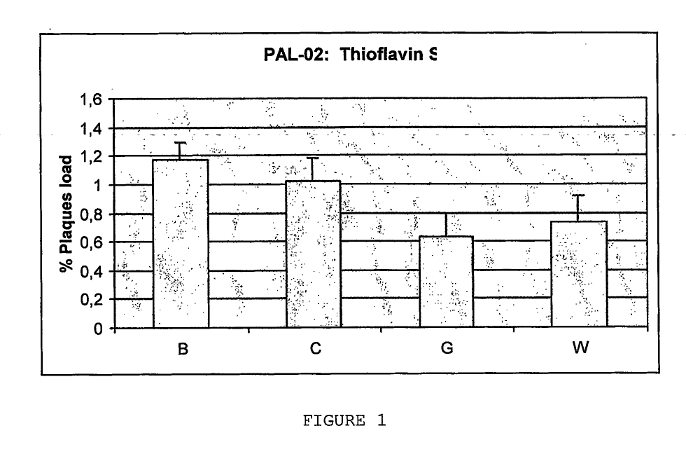

The invention relates to the use of nitrogeneous polycyclic derivatives for preparing drugs for treating neurodegenerative diseases. Said derivatives are useful as ligands to form complexes with transition metals, and the invention also relates to the use of such derivatives containing ligands as active principles.

Many studies have recently shown the major role of metal ions (copper, zinc, iron, . . . ) in modification of the folding or the aggregation of proteins, leading then to serious pathologies. Several neurodegenerative diseases (Alzheimer's disease, Parkinson and Huntington diseases, spongiform encephalopathies, . . . ) involve these disastrous non-desired interactions between metal ions and proteins.

In the case of Alzheimer disease, the pathology is associated with the aggregation of □-type amyloid peptides in the brain, leading to the formation of amyloid plaques. The accumulation of redox active metal ions in these amyloid plaques is deemed to be responsible for oxidative stress inducing neuronal lesions in the brain which result in irreversible loss of intellectual faculties.

The use of a ligand of metal ions like Clioquinol led to improvements in Alzheimer's disease indicating that therapeutic approaches are possible with metal ion chelators in neurodegeneratives diseases.

Recent works of the inventors on phenanthroline derivatives (“Phen” will be used to designate 1,10-phenanthroline) has demonstrated the benefit of complexing copper with two phenanthroline ligands connected to each other. It was therefore decided to prepare new cyclic uncharged ligands called “Cyclo-Phen”, small and sufficiently hydrophobic to be able to cross the barriers (first the intestinal barrier and then the blood brain barrier to go to coordinate the metal ions (copper in preference) which are present in excess in the pathogen proteins.

The invention thus relates to the use of nitrogeneous polycyclic derivatives for preparing drugs for treating neurodegenerative diseases, said derivatives having formula (I) 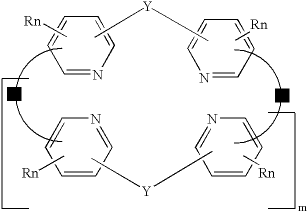
wherein
   Rn is anyone of R1, R2, R3 and R4, which are identical or different and represent H or represent one or several radicals and are selected in the group comprising —OH, an alkyl radical, —O-alkyl group, —NH₂, —NH-alkyl, —N (R5, R6), the alkyl being in said radical or groups a C1-C6 alkyl, or an halogen selected between the group consisting of F, Cl, Br,
   Y
      forms a phenyl group with both pyridines, optionally ortho-substituted by a substituent R5, or ortho-disubstituted by R5 and R6, said substituents being identical or different, and selected in the group comprising an alkyl radical, —O—alkyl group, —NH₂,—NH-alkyl, —N (R5, R6), the alkyl being in said radical or groups a C1-C6 alkyl, or an halogen selected between the group consisting of F, Cl, Br, or
      represents a group —(CH₂)ₘ₁—W—(CH₂)ₘ₁—, with m1 and m2 being 0, 1 or 2, and W being a group —CH₂—, —CH (R7), O, or N (R8, R9), R7, R8 and R9, identical or different, being a C1-C3 alkyl radical, or H,
   Z is a linking arm of formula -A-(CH₂)ₙ-U-(CH₂)ₙ-A-, 'A being O or NH, and
      U being selected in the group comprising —(CH₂)ₙₗ—, —N (R1,R2), —COOH, —OH,
with n being a number from 2 to 6, preferably from 2 to 4, and n1 being 0 or 1,
and the complexes thereof with transition metals, particularly with copper, zinc or iron.

According to an embodiment of the invention, said derivatives include 2 cyclic moieties.

According to another embodiment of the invention, said derivatives include 3 cyclic moieties.

According to still another embodiment, said derivatives include 4 cyclic moieties.

Preferably, the cyclic moieties consist of Phen moieties.

The invention particularly relates to the use of polycyclic Phen derivatives having formula (II)

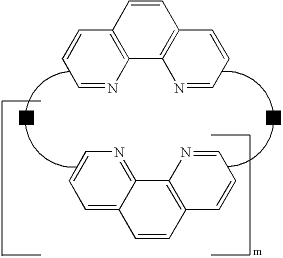

The invention particularly relates to the use of derivatives having 2, 3 or 4 Phen moieties.

The invention also relates to a method for the preparation of said derivatives.

The method of the invention comprises reacting
a dihydroxy bipyridine derivative of formula (III) 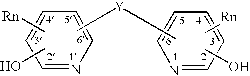
   with
a ditosyl derivative of formula (IV) 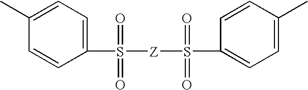
   wherein Rn, Y and Z are as above defined.

The reaction is carried out with high dilution conditions to limit oligomerizations.

The precursor of formula (III) is preferably used at concentrations of 0.1 to 20 mM in a polar solvent, such as DMSO.

In order to avoid β-elimination reactions, a weak base like cesium carbonate is used.

The derivatives of the invention have a low molecular weight (MW of 504 for the cyclic bi-Phen) and are poorly charged. Therefore they are able to cross the blood brain barrier in both directions (the metal ions present in excess in the pathogen proteins have to be chelated and the resulting complex has to be exported towards the blood circulation conducting to its ultimate excretion), Their structure can be altered to adjust the chelation selectivity in order to target certain metal ions.

It results from the pharmacological studies carried out with said derivatives that they have new activity spectrum and are particularly appropriate for the treatment of neurodegenerative diseases as above mentioned.

The invention relates to the use of said derivatives for preparing drugs for treating degenerative diseases comprising Alzheimer, Parkinson, Huntington diseases.

Said drugs comprise an effective amount of at least one derivative as above defined, associated with a pharmaceutical inert vehicle.

Said drugs are administered by the oral, intramuscular and intravenous route.

For oral administration, the drugs are presented in the form of tablets, pills, capsules or drops, patch, spray.

For administration by injection, the drugs are under the form of solution for injection by the intravenous, subcutaneous or intramuscular route produced from sterile or sterilisable solution, or suspension or emulsion.

The invention also relates to the use of said nitrogeneous polycyclic derivatives as chelating agents of transition metals.

Other characteristics and advantages of the invention will be given in the following examples given for illustrative purposes.

Cyclo-Phen preparation

Bromydrate of 3,8-dihydroxy-1,10-phenanthroline was synthesized through a method optimized in the laboratory (C. Boldron, M. Pitiéand B. Meunier, Synlett., 2001, 1629-1631). All the other commercially available reagents and the solvents were used without further purification. The NMR-spectra were recorded on a Bruker 250 MHz apparatus. The mass spectrometer used is a Perkin-Elmer SCIEX API 365 one and the analyses were done in positive mode. The UV-visible spectra were recorded with a Perkin-Elmer Lambda 35 spectrophotometer. Syntheses were monitored by thin-layer silica chromatography (on MERCK 60 F254 TLC aluminium sheets) eluted by CH₂Cl₂/CH₃OH (9/1, v/v) to which 1% of concentrated aqueous ammonia (30%) had been added, and spots were monitored under UV light (violet spots at 254 nm).

Cyclo-Phen synthesis: 2.22 g (6.83 mmol) of cesium carbonate were added to a solution of 0.40 g (1.37 mmol) of 3,8-dihydroxy-1,10-phenanthroline hydrobromide dissolved in 310 mL of anhydrous dimethylsulfoxyde (DMSO). Then a solution of 0.53 g (1.37 mmol) of 1,3-propanediol di-para-tosylate in 80 mL of anhydrous DMSO was added over 1 hour before to heat the mixture 48 hours at 50° C. under nitrogen and vigorous stirring. The volume was reduced to 100 mL then 40 mL of 30% aqueous ammonia were added and cyclized products were extracted with two volumes of CH₂Cl₂. The organic phase was washed with aqueous ammonia (pH=10) then evaporated before to be dried under vacuum. A chromatography on silica gel (eluent 1% triethylamine (TEA) in CHCl₃) afforded Cyclo-bi-Phen (31 mg, 0.06 mmol, yield=9%) as a white powder. A mixture of Cyclo-tri-Phen and Cyclo-tetra-Phen was then eluted from the column with CHCl₃/TEA/CH₃OH (94/5/1, v/v/v). After evaporation of the solvent, the two products were dissolved in CHCl₃/CH₃OH (9/3) then Cyclo-tetra-Phen was precipitated by addition of 6 volumes of CH₃OH. The supernatant was evaporated and a flash chromatography on silica gel (eluent 1% TEA in CHCl₃) afforded Cyclo-tri-Phen (14 mg, 0.013 mmol, yield=3%) as a white powder. Pure Cyclo-tetra-Phen was obtained from recrystallisation in hot CHC1₃/CH₃OH (3/1) as white crystals (10 mg, 0.01 mmol, yield=3%).

Cyclo-bi-Phen: ¹H NMR (250 MHz, in CDCl₃/CD₃OD: 3/1) □, ppm: 2.12 (m, 4H), 4.15 (m, 4H), 4.35 (m, 4H), 6.98 (d, ⁴J=3 Hz, 4H), 7.19 (s, 4H), 8.21 (d, ⁴J=3 Hz, 4H). ¹³C NMR (62.9 MHz in CDCl₃/CD₃OD 3/1) □. ppm: 153.3, 141.9, 138.2, 127.1, 126.6, 115.4, 63.4, 30.4. Mass spectrometry, electrospray, m/z: 505 (MH⁺). Elemental analysis: C₃₀H₂₄N₄O₄.0.6H₂O: % theoretical: C; 69.92, H; 4.93, N; 10.87; % found.: C; 70.01, H; 4.94, N; 10.53. UV-vis (H₂O/CH₃OH: 9/1): 237 nm (□=105000 mol⁻¹ cm^{31 1}), 281 (29500), 301 (18500), 319 (15000), 338 (9300), 355 (7200).

Cyclo-tri-Phen: ¹H NMR (250 MHz, in CDCl₃/CD₃OD: 3/1) □, ppm: 2.21 (quint, ³J=5 Hz, 6H), 4.20 (t, ³J=5 Hz, 12H), 7.26 (d, ⁴J=3 Hz, 6H), 7.36 (s, 6H), 8.50 (d, ⁴J=3 Hz, 6H). Mass spectrometry, electrospray, m/z: 757 (MH⁺). Elemental analysis: C₄₅H₃₆N₆O₆.CHCl₃: % theoretical: C; 63.05, H; 4.23, N; 9.59; % found: C; 62.61, H; 4.57, N; 9.01. UV-vis (H₂O/CH₃OH: 1/9): 241 nm (□=147000 mol⁻¹ cm⁻¹), 280 (44000), 300 (28500), 313 (23000), 339 (11500), 355 (11000).

Cyclo-tetra-Phen: ¹H NMR (250 MHz, in CDCl₃/CD₃OD: 3/1) . . . □, ppm: 2.31 (m, 8H), 4.20 (m, 16H), 7.37 (d, ⁴J=3 Hz, 8H), 7.49 (s, 8H), 8.54 (d, ⁴J=3 Hz, 8H). Mass spectrometry, electrospray, m/z: 1009 (MH⁺). Elemental analysis: C₆₀H₄₈N₈O₈.2 CHCl₃: % theoretical: C; 59.68, H; 4.04, N; 8.98; % found: C; 59.78, H; 3.62, N; 8.56. UV-vis (H₂O/CH₃OH: 9/1+4 HCl): 240 nm (ε=140000 mol⁻¹ cm⁻), 283 (53000), 301 (shoulder, 41000), 340 (16000), 356 (14500).

Complexation Properties of Cyclo-Bi-Phen, Cyclo-Tri-Phen and Cyclo-Tetra-Phen Derivatives in the Presence of CuCl₂

The complexes were studied by UV—visible spectroscopy and electrospray mass spectrometry.

The formation of a metallic complex resulted in a change of the absorption spectrum of the metallic ion and of the ligand.

Each Cyclo Phen was titrated by CuCl₂ to determine the maximal stoechiometry of the Cu complexes which were formed under the experimental conditions.

The studies were carried out between 200 and 420 nm at waves lengths involving the ligand orbitals, The 3 ligands were used in H₂O/MeOH at 10-20 μM. A solution of CuCl₂ at 2 mM was used in order to avoid variations of volume of more than 10% the initial volume.

Cyclo bi-Phen was solubilized in methanol/eau: 9/1 at a concentration of 14 μM. The maximal absorption band of the ligand at 237 nm and is submitted to a bathochrome and hypochrome effect during the complexation, a band with a maximal absorption at 345 nm being formed. The complexation with CuCl₂ results in the formation of various complexes during the addition of CUCl₂. Cyclo-tri-Phen was solubilized in methanol/eau: 9/1 at a concentration of 20 μM.5 isobestic points were observed at 227, 248, 283, 297 and 320 nm.

Preliminary Toxicity Studies on Mice with Three Different Chelating Agents

3-Propyl-Clip-Phen (M=432 Da; preparation according to C. Boldron et al., Synlett, 2001, 1629-1631), Cyclo-bi-Phen (M=504 Da; preparation as described in the present patent application) and Clioquinol (M=305; 5-chloro-7-iodo-8-hydroxyquinoline, purchased from Sigma).

These three compounds were tested on wild-type male FVB mice having a mean weight of 25 grams at 10 mg/kg by intraperitoneal (i.p.) injection at three consecutive days. At day 4, the animals were sacrified and checked for possible anatomical problems. The drugs were initially dissolved in DMSO in the presence of 2.6 equivalents of HCl and then diluted in water.

At 10 mg/kg, all mice survived at day 4 and no anatomical problems have been observed on stomach, spleen, kidneys, liver, heart, lungs and peritoneum. 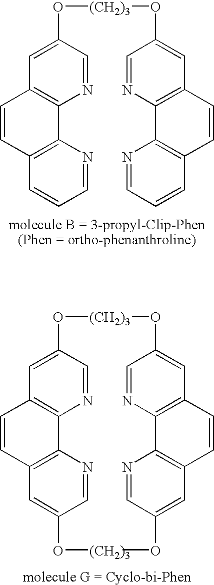
Experiments with these three Chelating Agents with Double Transgenic Mice Model of Alzheimer's Disease (AD).

Mice over-expressing human APP with the London mutation (V717I) and human PS1 bearing the A242E mutation (APP and PS1 stand for amyloid protein precursor and preseniline 1, respectively) were used. These animals develop many of the the pathological features of AD, including extensive deposition of amyloid plaques, neuritic dystrophy and astroglyosis (animals were identical to that used in the study performed by B. Permanne et al., FASEB J., 2002, vol. 16, 860-862).

Three molecules were evaluated on these double transgenic mice (6-month old): 3-Propyl-Clip-Phen (molecule B in the histogram below), Cyclo-bi-Phen (molecule G) and Clioquinol (molecule W) (C stands for control, only DMSO diluted in water). Clioquinol has already been used in the treatment AD transgenic mice by Cherny et al., Neuron, 2001, vol. 30, 665-676).

The molecules were initially diluted in DMSO in the presence of 2.6 equivalents of HCl and then in water and the animals were treated by i.p. injection with the two Phen derivatives at 5 mg/kg or at 10 mg/kg for Clioquinol, three times per week (monday, Wednesday and friday) during 9 consecutive weeks. 9 animals were treated for each drugs (control also included 9 animals). During the 9-week period, one animal was lost in each treatment group and none in the control group.

After 9 weeks of treatment, the animals were sacrified and the amyloid plaque loading brain sections was analyzed by staining with thioflavin S according to the protocol described by K. R. Bales et al., Nature Genetics, 1997, vol. 17, 263-264. This method is used to quantify the “old” plaques.

The histogramm below indicate that one Phen derivative, 3-Propyl-Clip-Phen has a negative effect: the plaque loading increased by 16%, whereas Cyclo-bi-Phen is able to reduce the plaque loading by 38%. In the same conditions, the reduction of Clioquinol is only 28%. Taking in consideration, the difference of molecular weight of these two chelators (504 for Cyclo-bi-Phen and 305 for Clioquinol), the 38% reduction has been obtained with 9.9 micromoles/kg with Cyclo-bi-Phen and 32.8 micromoles/kg with Clioquinol, a drug charge 3.3 times higher.

These data obtained on the reduction of thioflavin-S stained amyloid desposit is of particular interest since these thioflavin-staine plaques are now considered as being selectively neurotoxic (see B. Urbanc et al., PNAS, 2002, vol. 99, 13990-13995).

This significative reduction of the plaque loading observed with Cyclo-bi-Phen clearly indicate that the Cyclo-Phen derivatives can be considered as drug candidates in the treatment of neurodegenerative diseases where an over-loading of metal ions in brain have been evoked as being one of the main factors of the pathologies such as Alzheimer's disease, Parkinson's disease and any other pathologies related to metal-related misfolding of proteins (Huntington's disease and spongiform encephalopathies).

1-15. (canceled)

16-32. (canceled)

33. A drug composition comprising at least one nitrogeneous polycyclic derivative of the following formula 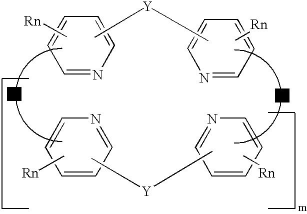
wherein
m=1, 2 or 3
Rn is anyone of R1, R2, R3 and R4, which are identical or different and represent H or represent one or several radicals and are selected in the group comprising OH, an alkyl radical, O-alkyl group, NH₂, NH-alkyl, N(R5, R6), or an halogen selected in the group consisting of F, Cl, Br, the alkyl being in said radical or groups, a C1-C6 alkyl, R₅ and R₆ being a C1-C3 alkyl group,
Y forms a phenyl group with both pyridines, optionally ortho-substituted by a substituent R7, or ortho-disubstituted by R7 and R8, said substituents being identical or different, and selected in the group comprising an alkyl radical, 0-alkyl group, NH₂, NH-alkyl, N(R5, R6), or an halogen selected between the group consisting of F, Cl, Br, the alkyl being in said radical or groups a C1-C6 alkyl and R₅ and R₆ are as above defined
or
Y represents a group —(CH₂)ₘ₁—W—(CH₂)m₂—, with m1 and m2 being 0, 1 or 2 and W being a group CH₂, CH(R9), O, or N(R10), R9 and R10 being a C1-C3 alkyl radical, or H,
Z is a linking arm of formula -A-(CH₂)n-U-(CH₂)n-A-
wherein
A being O or NH, and
U being selected in the group comprising (CH₂)ₙ₁, CHN(R5,R6), CHCOOH, CHOH
with n being a number from 1 to 6, preferably from 2 to 4, and n1 being 0 or 1,
and the complexes thereof with transition metals, particularly with copper, zinc or iron.

34. The composition according to claim 33, wherein said derivative includes 2 cyclic moieties.

35. The composition according to claim 33, wherein said derivative includes 3 cyclic moieties.

36. The composition according to claim 33, wherein said derivative includes 4 cyclic moieties.

37. The composition according to claim 33, wherein, in said derivative, the cyclic moiety consists of a Phen moiety.

38. The composition according to claim 37, wherein said derivative is a polycyclic Phen having formula (II)

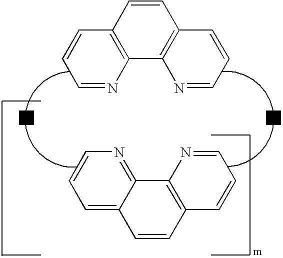

39. The composition according to claims 33, in a form for treating degenerative diseases selected from Alzheimer disease, Parkinson disease, and Huntington disease.

40. The composition according to claim 33, wherein the composition comprises an effective amount of at least one derivative with a pharmaceutical inert vehicle.

41. The composition of claim 40, in an oral, intramuscular or intravenous administerable form.

42. The composition according to claim 41, in the form of tablets, pills, capsules, drops, patch, or spray.

43. The composition according to claim 41, in the form of an injectionible solution produced from sterile or sterilisable solution, or suspension or emulsion.

44. A method for preparing the derivatives of claim 33, comprising reacting
a dihydroxy bipyridine derivative of formula (III) 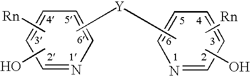
with
a ditosyl derivative of formula (IV) 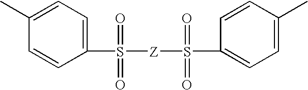
wherein Rn, Y and Z are as defined in claim 33.

45. The method of claim 44, wherein the reaction is carried out with high dilution conditions.

46. The method of claim 44, comprising the use of cesium carbonate.

47. A method of chelating transition metals comprising contacting a composition of claim 33 with a material comprising said metals.

48. A method of treating a neurodegenerative disease comprising administering a composition of claim 33 to an individual in need of said treatment.

49. The method of claim 48 wherein said disease is selected from Alzheimers disease, Parkinsons disease and Huntingtons disease.
